(19) United States
(12) Patent Application Publication
(10) Pub. No.: US 2023/0043147 A1
(43) Pub. Date: Feb. 9, 2023

(54) MULTIWELL, MICROSCOPE-COMPATIBLE DEVICE FOR HIGH-THROUGHPUT ANALYSIS OF CELL INVASION

(71) Applicant: UNIVERSITÄT ZÜRICH, Zürich (CH)

(72) Inventors: Karthiga Santhana KUMAR, Zürich (CH); Michael GROTZER, Zürich (CH); Martin BAUMGARTNER, Rubigen (CH)

(73) Assignee: UNIVERSITÄT ZÜRICH, Zürich (CH)

(21) Appl. No.: 17/783,658

(22) PCT Filed: Dec. 9, 2020
(86) PCT No.: PCT/EP2020/085366

§ 371 Date: Jun. 9, 2022

(30) Foreign Application Priority Data
Dec. 9, 2019 (EP) 19214586.0

(51) Int. Cl. B01L 3/00

(52) U.S. Cl. CPC B01L 3/5085; B01L 2300/0829; B01L 2300/161; B01L 2300/0851

(57) ABSTRACT

The invention is related to a device (1) for receiving a biological sample (3) wherein the device (1) comprises a plurality of wells (10) wherein each well (10) comprises an inner surface (14) facing a volume (60) for receiving a biological sample (3). The inner surface (14) comprises a top section (20) and a bottom section (30). The top section (20) and the bottom section (30) are connected via a circumferential step (40). The circumferential step (40) forms a stop for a tip of a pipette (70).

The invention relates to device for receiving a biological sample. In particular, the device is configured to receive a microtissue, in particular a spherical microtissue. A microtissue is a 3-dimensional aggregate of cells. It can be produced by self-aggregation of cells to spheroids wherein a microtissue particularly comprises 500 to 10000 cells, particularly adherent cells.

For instance, a microtissue can be used to investigate cell migration, in particular cell invasion. Cell invasion refers to a process in that a cell becomes motile and can move through the extracellular matrix of a tissue or might get in an adjacent tissue. With regard to cancer cell dissemination and development of metastases, a deeper understanding of cell migration, in particular cell invasion, is of growing importance and appropriate devices and methods for receiving and analyzing microtissues are needed.

One out of two approaches is commonly used for the generation of a microtissue: either the hanging drop method or the generation using a microwell plate.

In the hanging drop method, a microtissue is generated at the bottom of a hanging drop. After the microtissue is generated it has to be transferred to another device for a further analysis. Hence, the hanging drop method requires separate devices for the generation of the microtissue and its subsequent analysis. This increases time and material requirements.

In an alternative approach, a microtissue can be generated using a microwell plate. In particular, the bottom of each well is coated such that the adhesion of the cells to the well is prevented. An adhesion of cells to the well would have a negative effect on the microtissue generation.

The generation of a microtissue using a microwell plate requires special attention, in particular when a medium is removed or added to a well containing cells such that the respective cells are neither disturbed nor aspirated.

In GB2539935A, a device for propagating microtissues is disclosed that comprises a pipetting stage. The device is configured such that a fluid applied to the pipetting stage can get from the pipetting stage to the bottom part of a well in which a microtissue can be located. In particular, the device is configured such that the fluid gets from the pipetting stage to the bottom part in a way that the microtissue in the bottom part is not disturbed.

For an analysis of the cell invasion, a microtissue can be embedded in a polymerized matrix and the localization of cells can be observed, in particular an alteration of the localization of a plurality of cells. For a proper comparison of different experiments and the reproducibility of an experiment, the knowledge of the density of the polymerized matrix is of high importance. Amongst others this is also the basis for a quantitative analysis and/or high-throughput analyses of cell invasion.

Hence, there is a need to provide a device for receiving a biological sample. In particular, there is a need to provide a device for receiving and generating a biological sample in an easy, secure and cost-efficient manner, and wherein particularly the device can be used for a subsequent analysis of the respective biological sample.

This problem is solved by a device according to claim 1 and a method according to claim 25. Advantageous embodiments of the respective aspect of the present invention are stated in the corresponding sub claims.

A first aspect of the invention is related to a device for receiving a biological sample, wherein the device comprises a plurality of wells. Each well comprises an inner surface facing a volume for receiving a biological sample. The inner surface comprises a top section and a bottom section, wherein the top section and the bottom section are connected via a circumferential (e.g. annular) step. The circumferential step forms a stop for a tip of a pipette. Particularly, the step extends along the entire circumference of the respective well.

Each well can comprise an opening of the well wherein the opening is configured such that the volume can be accessed through the opening of the well. The top section can comprise an upper edge that delimits the opening of the well. According to an embodiment of the device, the opening of the well is spaced apart from the bottom section.

Each well can extend along a central axis. According to an embodiment, the central axis can extend perpendicular to the opening of the respective well.

In an embodiment, the top section can have the shape of an open cylinder. In another embodiment, the top section can comprise four rectangular sides.

In an embodiment each well of the plurality of wells of a device is configured identically.

In an embodiment, each well tapers from the top section towards the bottom section. The circumferential step can comprise an inner edge and an outer edge wherein the outer edge is located adjacent to the top section and the inner edge is located distant to the top section. In other words this means that with respect to the top section the circumferential step is directed inwards, i.e. towards the central axis.

The inner edge and the outer edge can be continuous lines. In an embodiment, the inner edge and/or the outer edge can have a circular shape. In another embodiment, the inner edge and/or the outer edge can have a rectangular shape, in particular a square shape.

Further, the circumferential step of a well can extend along the entire circumferential direction of the respective well and can form a circumferential surface. Particularly, it can form a stop for a tip of a pipette. In an embodiment, a tip of a pipette can be the tip, i.e. a front end, of a single-use tip of a pipette. A tip of a pipette comprises a face side. The face side can comprise an edge of a pipette that delimits an opening of the tip of the pipette, also referred to as opening of the pipette. Through the opening of the pipette a fluid can be drawn up into the pipette or can be released from the pipette.

The tip of a pipette can be positioned on the circumferential step. Particularly, the circumferential step is configured to contact and support at least a part of the face side of the pipette, particularly to prevent a further penetration of the pipette into the respective well.

In an embodiment, the inner edge of the circumferential step marks a predefined lower portion of the volume. In particular, the inner edge marks an upper boundary of the lower portion of the volume. Further, the lower portion is determined by the bottom section. In an embodiment the bottom section and the circumferential step are arranged and configured such that the lower portion of the volume is in the range from 10 μl to 120 μl, in particular from 15 μl to 50 μl, in particular 30 μl.

In an embodiment, the lower portion of the volume is equal in each well of the plurality of wells of the device.

Another embodiment is characterized in that the bottom section comprises a concave curvature. In an embodiment according to the invention, the entire bottom section is concavely curved. In an embodiment, the bottom section comprises a lowest point. Particularly, the lowest point is characterized in that the distance from the lowest point to the opening of the well parallel to the central axis is greater than the distance from any other point of the bottom section to the opening of the well parallel to the central axis.

A biological sample can comprise a plurality of cells. Cells that are put into a well can particularly accumulate in the bottom section and can generate a cluster of cells or a spheroid of cells, in particular a spheroid of tumor cells. The concave curvature of the bottom section promotes the accumulation of the cells close to the lowest point. The concave curvature of the bottom section promotes a central localisation of the cells. In other words, the concave curvature can promote a central accumulation of the cells, i.e. an accumulation close to the central axis of the respective well. Further, the concave curvature prevents an accumulation of cell at or close to the inner edge of the circumferential step which borders the bottom section. The central localisation of the cells (relative of vertical axis of well) is in advantage for monitoring cell invasion after matrix embedding.

In an embodiment, the bottom section comprises a spherical curvature.

Further, in an embodiment, the bottom section is spherically curved. Furthermore, according to an embodiment, the bottom section can comprise the shape of a segment of a spheroid or a sphere.

Further, the bottom section can have the shape of a hemisphere or a semi-spheroid wherein the semi-spheroid can be flattened or elongated.

In an embodiment, a well, some wells or all wells are configured such that the lowest point is located on the central axis.

Cells that are put into a well can particularly accumulate close to the lowest point. Consequently, in an embodiment in which the lowest point is located on the central axis, the cells can accumulate close to the central axis. In this way, an accumulation of the cells at or close to the inner edge of the circumferential step can be prevented. Cells, particularly spheroids, located close to the wall of a well are difficult to be imaged using microscopy.

Cells that are centrally located can be easily accessed such that a subsequent investigation can be easily performed. In particular, the centrally located cells are visually easy to access.

The bottom section can be configured to optimise for spheroid formation. In particular, the shape, the curvature and/or the radius of the bottom section can be configured to optimise for spheroid formation.

In an embodiment, the bottom section is configured such that spheroid formation is provided and the visual assessment of the cells, in particular by means of a microscope, is provided.

In another embodiment, the bottom section comprises a flat bottom. The flat bottom is located distant to the opening of the well.

The flat bottom can extend in a first plane. In an embodiment, the first plane extends perpendicular to the central axis.

In an embodiment, the bottom section comprises a lateral portion connected to the flat bottom, particularly at an angle. In an embodiment, the lateral portion connects the flat bottom and the circumferential step.

The lateral portion can comprise an upper and a lower end, wherein the lower end is located more distant to the opening of the well than the upper end. With its lower end, the lateral portion can be (e.g. integrally) connected to the flat bottom, while the upper end of the lateral portion can adjoin the circumferential step, in particular the inner edge of the circumferential step.

In an embodiment, the lateral portion is cylindrical.

The lateral portion can extend parallel or coaxially to the central axis such that the cylinder is a right cylinder. In an embodiment, the cylinder is a right circular cylinder.

In an alternative embodiment, the lateral portion comprises four rectangular sides.

In an embodiment, each two adjacent rectangular sides are arranged perpendicular to each other. Further, rectangular sides that face each other can comprise the same shape. Furthermore, in an embodiment, each of the four rectangular sides comprises the same shape. Particularly, this means that each of the four rectangular sides has the same height and width.

In an embodiment, a first angle enclosed by the top section and the circumferential step is in the range from 90° to 135°, in particular from 100° to 130°, in particular from 115° to 125°. In particular the first angle amounts to 120°.

In an embodiment, the top section and the circumferential step are arranged perpendicular to each other. In a case the top section extends parallel or coaxially to the central axis, the circumferential step can extend in a second plane that is located perpendicular to the central axis. Here, both the outer edge and the inner edge are located in the second plane. In an embodiment, the inner edge and the outer edge can have a circular shape and can be arranged such that the circumferential step can have the shape of a circular ring. In another embodiment the inner edge and the outer edge can have a rectangular shape. In another embodiment, the circumferential step can comprise a rectangular outer edge and a circular inner edge.

In an alternative embodiment, the first angle is an obtuse angle, in particular an angle in the range between 100° and 130°. In case the top section extends parallel or coaxially to the central axis, the circumferential step extends away from the top section, in particular away from the opening of the well. This means that the outer edge is located on a second plane and the inner edge is located on a third plane wherein the second and the third plane extend perpendicular to the central axis and wherein the second plane is located closer to the opening of the well than the third plane. The circumferential step can have a conical shape. In particular, the circumferential step can comprise the shape of a right circular cone. In another embodiment, the circumferential step can have the shape of a frustum, in particular of a right frustum, in particular of a square frustum.

The circumferential step can be configured such that the circumferential step directs an object put on the circumferential step, e.g. a cell, towards the bottom section, in particular towards the lower portion of the volume.

According to an embodiment, each well extends along a longitudinal axis, wherein the circumferential step is configured such that the circumferential step encloses an acute angle with the longitudinal axis.

In an embodiment, the acute angle lies in the range from 5° to 85°, in particular in the range from 15° to 75°, in particular in the range from 30° to 60°.

The circumferential step can be decline towards the bottom section. The circumferential step can be decline towards the lower portion of the volume.

An advantage of a circumferential step declining towards the bottom section is that an object that has been put on the circumferential step is directed by the circumferential step towards the bottom section, particularly towards the lower portion of the volume of the respective well. In an embodiment, a cell that has been positioned on the circumferential step by means of an applicator (e.g. a pipette) can slide along the circumferential step into the lower portion of the volume. This advantageously supports the formation of the microtissue since all applied cells are directed towards the lower portion of the volume (the bottom section).

Additionally, a fluid applied on the circumferential step declining towards the bottom part can advantageously flow in the bottom part.

In an embodiment the circumferential step is configured such that a cell can be guided from the circumferential step towards the lower portion of the volume while maintaining the function of the circumferential step to form a stop of the pipette.

In an embodiment, each well extends along a central axis and is rotationally symmetrical with respect to the respective central axis.

The central axis can be the longitudinal axis.

In an embodiment, the circumferential step is configured to enclose the acute angle with the central axis.

Further, in an embodiment, the top section can have the shape of an open cylinder.

In another embodiment, each well comprises a transparent material.

In particular, at least the bottom section of each well comprises or is formed out of a transparent material. In an embodiment in which the respective well comprises a flat bottom, said flat bottom can be formed out of a transparent material.

In a further embodiment each well is formed out of a transparent material.

The transparent material can be a plastic or a polymer. In particular, the transparent material can be polystyrene, polyvinyl chloride or polypropylene. In an alternative embodiment, the transparent material can be a glass.

Due to the transparent material, a biological sample in a well is visually easy to access by means of a microscope.

In an embodiment, the device is configured to be arranged on a stage of a microscope.

In particular, the device is configured such that the device can be arranged on a stage of a fluorescence microscope.

Hence, the device that receives the biological sample can also be used for a further analysis of the respective biological sample, in particular with respect to the visualization and/or the imaging of the biological sample by means of a microscope.

In an alternative embodiment the bottom section comprises a coating for reducing adhesion of the biological sample.

By means of the coating the adhesion of the biological sample to the bottom section is reduced, in particular the adhesion is prevented. It is an advantage to reduce adhesion because adhesion can negatively affect the accumulation of cells, i.e. a formation of a cell cluster, in particular a formation of a microtissue, in particular the formation of a spheroid. In other words, this means that by means of the coating microtissue formation is promoted.

The coating can comprise agar, water-soluble polymer of 2-methacryloyloxy ethyl phosphorylcholine (MPC), commercially available as Lipidure®. In an alternative embodiment, the coating can comprise a covalently bound hydrophilic, non-ionic, neutrally charged hydrogel.

In an embodiment, the respective well comprises the bottom section comprising a concave curvature, the circumferential step and super low adhesion coating. This can advantageously support spheroid cell growth, because cells are directed towards the lower portion of the volume. The cells can centrally accumulate in the lower portion of the volume. The cells can accumulate at the lowest point of the respective well and form spheroids.

In an embodiment, the device comprises at least 6, particularly at least 10, particularly at least 20, particularly at least 30, particularly at least 40, particularly at least 50, particularly at least 60, particularly at least 70, particularly at least 80, particularly at least 90, particularly at least 96 wells wherein the plurality of wells forms an array of wells.

In particular, the wells of the array of wells can be arranged in a format of a rectangular matrix with a row-to-column-ratio of 2 to 3. In an embodiment, the wells are arranged such that the array comprises 8 rows wherein each row comprises 12 wells.

In an embodiment, the device is a standard SBS (Society of Biomolecular Screening) format multiwell plate. The device can have a length of 127 mm to 128 mm and a width of 85 mm to 86 mm.

According to an embodiment, the device can be compatible with a multichannel pipette, in particular with an 8-channel-pipette or with a 12-channel-pipette. A multichannel pipette comprises a plurality of tips of a pipette. An 8-channel-pipette or a 12-channel-pipette can comprise eight or 12 tips of a pipette, respectively. Further, the device can be compatible with a 96-channel-pipette comprising 96 tips of a pipette.

In particular, the wells of the device can be arranged such that a plurality of channels, i.e. a plurality of tips of the pipette, can simultaneously enter a plurality of wells. This advantageously reduces the time and effort required. Additionally, the possibility is provided to use the device in a high-throughput method that comprises the use of a multichannel pipette.

According to an embodiment, the top section comprises a lower top section and an upper top section, wherein the lower top section and the upper top section are connected via a further circumferential step.

Particularly, the respective well can be configured such that the lower top section and the bottom section are connected via the circumferential step.

The upper top section can comprise the upper edge that delimits the opening of the well.

According to the invention, the respective well can comprise a circumferential step and a further circumferential step.

The further circumferential step of a respective well can extend along the entire circumferential direction of the respective well. The further circumferential step can form a circumferential surface. The further circumferential step can form a further stop for a tip of a pipette.

The further circumferential step can comprise a further inner edge and the further outer edge. The further inner edge and the further outer edge can be continuous lines.

The respective well can be configured such that along the longitudinal direction, the circumferential step is distant to the further circumferential step.

In an embodiment, the further circumferential step enclosed a further acute with the longitudinal axis, in particular wherein the further acute angle is between 5° and 85°, in particular between 15° and 75°, in particular between 30° and 60°.

The further circumferential step can enclose the further acute angle with the central axis.

The further circumferential step can decline towards the lower top section. In an embodiment, the further circumferential step has a conical shape.

The further circumferential step can be configured such that it tapers from the further outer edge towards the further inner edge.

An embodiment is characterized in that the further circumferential step and the circumferential step run in parallel to each other.

In an embodiment, the acute angle and the further acute angle are identical.

The circumferential step can extend along a circumferential direction of the respective well. In particular, the circumferential step can extend uniformly along the circumferential direction. The further circumferential step can extend along a circumferential direction of the respective well. Particularly, the further circumferential step can extend uniformly along the circumferential direction.

In an embodiment, the further circumferential step comprises a further inner edge marking a predefined extended lower portion of the volume.

The predefined extended lower portion of the volume can comprise the predefined lower portion of the volume. The predefined extended lower portion of the volume can comprise a further portion of the volume.

According to an embodiment, the predefined extended lower portion of the volume consists of the predefined lower portion of the volume and the further portion of the volume.

The further portion of the volume can adjoin the predefined lower portion of the volume.

The further portion of the volume can be marked by the inner edge of the circumferential step and a further inner edge of the further circumferential step.

In an embodiment, the predefined lower portion of the volume is at least 10% of the volume, in particular at least 15% of the volume, in particular at least 20% of the volume.

In an embodiment, the predefined lower portion of the volume is 20% of the volume.

The volume can comprise an upper portion of the volume. In an embodiment, the volume consists of the predefined lower portion of the volume and the upper portion of the volume.

The upper portion of the volume can comprise a topmost portion of the volume. The topmost portion of the volume can comprise the opening of the respective well. In an embodiment, the topmost portion of the volume is at least 10% of the volume, in particular at least 15% of the volume, in particular at least 20% of the volume.

The predefined lower portion of the volume can equal the topmost portion of the volume. In an embodiment, the predefined lower portion of the volume is 20% of the volume and the topmost portion of the volume is 20% of the volume.

The topmost portion of the volume can determine a maximum fill level of the respective well.

In an embodiment, the predefined lower portion of the volume is at least 5% of the volume, in particular at least 7.5% of the volume, in particular at least 10% of the volume.

In particular, the predefined lower portion of the volume is at least 5% of the volume, in particular at least 7.5% of the volume, in particular at least 10% of the volume, when the respective well comprises the circumferential step and the further circumferential step.

According to an embodiment, the predefined extended lower portion of the volume is at least 20% of the volume, in particular at least 30% of the volume, in particular at least 40% of the volume.

In an embodiment, the predefined extended lower portion of the volume is 40% of the volume, wherein the predefined lower portion of the volume is at least 10% of the volume.

The upper portion of the volume can comprise the topmost portion of the volume, wherein the topmost portion of the volume can define a maximum fill level of the respective well. The topmost portion of the volume can be 20% of the volume.

The further circumferential step and the circumferential step can be arranged such with respect to each other that a specific predefined ratio between the lower portion of the volume and the extended lower portion of the volume is met. The further circumferential step and the circumferential step can be arranged such with respect to each other that a specific predefined ratio between the lower portion of the volume and the further portion of the volume is met. The further circumferential step and the circumferential step can be arranged such with respect to each other that a specific predefined ratio between the lower portion of the volume and the upper portion of the volume is met. The further circumferential step and the circumferential step can be arranged such with respect to each other that a specific predefined ratio between the lower portion of the volume and the topmost portion of the volume is met.

The further circumferential step and the circumferential step can be arranged such with respect to each other that a specific predefined ratio between the further portion of the volume and the extended lower portion of the volume is met. The further circumferential step and the circumferential step can be arranged such with respect to each other that a specific predefined ratio between the between the further portion of the volume and the upper portion of the volume is met. The further circumferential step and the circumferential step can be arranged such with respect to each other that a specific predefined ratio between the further portion of the volume and the topmost portion of the volume is met.

The further circumferential step and the circumferential step can be arranged such with respect to each other that a specific predefined ratio between the extended lower portion of the volume and the upper portion of the volume is met. The further circumferential step and the circumferential step can be arranged such with respect to each other that a specific predefined ratio between the extended lower portion of the volume and the topmost portion of the volume is met.

The further circumferential step and the circumferential step can be configured to establish pre-defined volume ratios in the different well compartments.

Advantageously, the circumferential step and the further circumferential step are arranged to provide an easy and user-friendly medium removal and/or medium exchange from matrix-embedded cultures.

According to an embodiment, the top section tapers towards the circumferential step. In an embodiment, the lower top section tapers towards the circumferential step. The lower top section can enclose a tapering angle with the longitudinal axis, wherein the tapering angle is an acute angle.

The tapering angle can be smaller than the acute angle enclosed by the circumferential step and the longitudinal axis. In an embodiment, the tapering angle is smaller than the further acute angle enclosed by the further circumferential step and the longitudinal axis.

In an embodiment, the upper top section runs parallel to the longitudinal axis and the lower top section tapers towards the circumferential step. The upper top section can extend coaxially to the central axis.

Along the longitudinal axis, the top section can taper between the circumferential step and the further circumferential step.

The circumferential step can have a circular shape. The circumferential step can be defined by a diameter. The further circumferential step can have a circular shape. The further circumferential step can be defined by a further diameter. In an embodiment, the further diameter is greater than the diameter.

The top section can be configured to guide the tip of the pipette. In an embodiment, the lower top section is configured to guide the tip of the pipette towards the circumferential step.

A further aspect of the invention is related to a method for propagating biological samples using a device according to the invention. The method comprises the steps of:
placing a suspension of cells in a culture medium into a plurality of wells of the device,
incubating the cells,
placing a tip of a pipette on the respective circumferential step, and
removing culture medium by means of the pipette such that culture medium and cells remain in the predefined portion of the volume of the respective well.

In particular, the method comprises the step of removing culture medium by means of the pipette such that culture medium and cells remain in the predefined lower portion of the volume of the respective well.

In an embodiment, a suspension of cells in a culture medium is placed into all wells of the device.

An embodiment is characterized in that tumor cells in a culture medium are placed into a plurality of wells. Tumor cells are also referred to as cancer cells. According to an embodiment, 1000 to 2000 cells, particularly tumor cells, are placed into each well. In an embodiment, cells in 80 μl to 120 μl culture medium, in particular 100 pl culture medium, are placed into each well of the plurality of wells.

In particular, cells accumulate in the lower portion of the volume of each well.

According to an embodiment, a tip of a pipette is placed on the circumferential step of the respective well. The pipette can be a pipette comprising one tip of a pipette. In an alternative embodiment the pipette can be a multichannel pipette comprising a plurality of tips of a pipette. In particular, the multichannel pipette can be an 8-channel-pipette or a 12-channel-pipette or a 96-channel-pipette, comprising eight or 12 or 96 tips of a pipette, respectively.

The tip of the pipette can be placed on the circumferential step such that at least a part of the face side of the pipette can touch the circumferential step.

The circumferential step can precisely stop the tip of the pipette such that the respective pipette cannot be further inserted into the respective well. In particular the tip of the pipette can be stopped by the circumferential step such that the tip of the pipette cannot enter the lower portion of the volume.

A pipette whose tip is positioned on the circumferential step can be used to remove culture medium from the respective well. In an embodiment, culture medium is removed until air instead of culture medium is aspirated by means of the pipette. This means that culture medium up to a marking is removed, in particular up to the marking that marks the predefined lower portion of the volume. This means that culture medium remains only in the lower portion of the volume. Hence, a predefined amount, i.e. volume, of culture medium can easily remain in a well, in particular in a plurality of wells. In an embodiment, a plurality of wells of the device are configured identically such that the lower portion of the volume of the plurality of wells are equal such that the same amount of culture medium can remain in each well of a plurality of wells of the device.

Further, by placing the tip of the pipette on the circumferential step it is prevented that the biological sample in the respective well, in particular in the lower portion of the respective well, is disturbed or aspirated by means of the pipette.

In an embodiment, the method is characterized in that a portion of the culture medium is replaced by fresh culture medium at least once or several times.

In particular this means that after removing culture medium until culture medium remains only in the lower portion, fresh culture medium is added to a plurality of wells. Afterwards, the cells can be incubated again.

The invention comprises that the culture medium is replaced once by fresh culture medium. In an alternative embodiment, the culture medium is replaced several times by fresh culture medium. In particular, culture medium is replaced 1 to 7 times.

By means of an embodiment of the method the biological sample, in particular cells, a cluster of cells or a cell spheroid, can be propagated for a longer time than a biological sample that is propagated without the exchange of the culture medium. The biological sample can be propagated up to 14 days.

In an embodiment the method further comprises the step of adding a compound to the culture medium in the respective well such that the culture medium polymerizes.

The compound that polymerizes the culture medium can be a mixture of biomolecules that can act as a base for polymer assembly. For instance, such a mixture is a matrigel mixture. In an alternative embodiment, the compound that polymerizes the culture medium can be collagen.

In an embodiment, the compound that polymerizes the culture medium is added after removing culture medium until culture medium remains only in the lower portion of the volume of the respective well. Hence, the compound that polymerizes the culture medium is added to a known amount of culture medium such that the quantity of the compound that polymerizes the culture medium can be adapted such that a polymerization of the culture medium to a predefined density is achievable. Hence, a predefined density of the polymerized matrix can easily be generated.

A polymerized matrix is particularly used to investigate the motility of cells, in particular cell invasion, by investigating the infiltration of the polymerized matrix by a cell.

For a quantitative analysis of the infiltration, the knowledge of the density of the polymerized matrix is required because the density of the polymerized matrix can have an impact on the cell motility.

In an alternative embodiment the method further comprises an addition of a solution to the culture medium in a plurality of wells wherein the solution is a control medium or the solution comprises one of: a stimulating compound for stimulating the biological sample, an inhibiting compound for inhibiting the biological sample or a combination of a stimulating and an inhibiting compound.

The control medium can be a basal medium without any supplemental additives.

The stimulating compound can particularly be a growth factor. In an embodiment, the stimulating factor is basic the Fibroblast Growth Factor (bFGF). In another embodiment, the stimulating factor is the Hepatocyte Growth Factor (HGF). Alternatively, the stimulating factor can be the Epidermal Growth Factor (EGF). The inhibitory compound can be a tyrosine kinase inhibitor such as e.g. ARQ197, PHA665752, and BGJ398. Alternatively, the inhibitory compound can be an actin polymerization inhibitor, for instance CK-666. The stimulating compound can be applied in combination with a tyrosine kinase inhibitor or an actin polymerization inhibitor.

A stimulating compound, an inhibiting compound or the combination of both can affect the motility of a cell. Hence, by means of an embodiment of the method, the motility of a cell exposed to a stimulating compound, an inhibiting compound or a combination of both can be analyzed. Additionally, the motility of a cell not exposed to an activating and/or inhibiting compound can be analyzed.

An embodiment of the invention is characterized in that the method further comprises the steps of adding a fluorescent dye to the culture medium in a plurality of wells, putting the device on the stage of a microscope, in particular a fluorescence microscope, and acquiring at least one image of the biological sample in the respective wells by means of the microscope.

The fluorescent dye can be added to the culture medium not polymerized by means of a compound to polymerize the culture medium. In an alternative embodiment the fluorescent dye can be added to polymerized culture medium. In particular, the fluorescent dye is added such that the fluorescent dye can get in the respective biological samples.

In an embodiment, the fluorescent dye is a fluorescent dye used to stain DNA. The fluorescent dye can be a bis-benzimide, in particular a Hoechst stain. In an alternative embodiment, the fluorescent dye is 4′,6-diamidino-2-phenylindole, also known as DAPI. Hoechst stain as well as DAPI can enter living cells.

The method comprises the use of a fluorescence microscope. By means of a fluorescence microscope structures stained with the fluorescent dye can be observed.

The fluorescence microscope can be a standard fluorescence microscope. In an alternative embodiment, the fluorescence microscope can be an automated fluorescence microscope. Further, the fluorescence microscope can be a semi-automated fluorescence microscope.

In particular, by means of the microscope, an image with an up to 640-fold magnification, in particular a 50-fold magnification can be acquired. Further, the microscope can be configured to process and/or store data, particularly to process and/or store an image.

In an embodiment the fluorescent dye can be added to culture medium that is not polymerized by means of a compound to polymerize the medium. Additionally, a control medium or a solution comprising a stimulating compound, an inhibiting compound or a combination of both can be added.

By means of the method, a viability of a cell can be detected. In particular it can be detected whether a cell is dead or alive.

In an alternative embodiment, the fluorescent dye can be added to a plurality of wells after the culture medium has polymerized. The fluorescent dye can enter the biological sample such that the cells are easy to access my means of the fluorescence microscope such that an infiltration of the polymerized medium by the cells can be easily observed. This means that cell invasion can be analyzed in a simple manner.

An embodiment of the invention is characterized in that the tip of the pipette comprises an opening wherein the tip of the pipette is placed such that the opening of the tip of the pipette and the circumferential step enclose a second angle. According to the invention the second angle is in the range from 0° to 75°, in particular from 5° to 45°.

The second angle is an acute angle.

By positioning the opening of the tip of the pipette and the circumferential step in an acute angle, a fluid can be easily drawn up into the pipette.

According to an embodiment of the method, the method comprises the steps of:
placing a suspension of cells in a culture medium into a plurality of wells of the device,
incubating the cells,
placing a tip of a pipette on the respective further circumferential step, and
removing culture medium by means of the pipette such that culture medium and cells remain in the predefined extended lower portion of the volume of the respective well.

In an embodiment, the medium in the lower portion of the volume is mixed with a matrix solution, wherein the matrix solution is filled in the further portion of the volume.

Hence, a predefined volume of matrix solution can easily be mixed with a medium in the lower portion of the volume. A desired concentration of the medium mixed with the matrix solution can be generated in the extended lower portion of the volume, in particular in the lower portion of the volume and the further portion of the volume. In particular, by polymerisation, a generated matrix is generated in the extended lower portion of the volume upon mixing the predefined volume of matrix solution and the medium in the lower portion of the volume. The generated matrix can have a particular predefined concentration. In an embodiment, the generated matrix can have a particular density.

According to an embodiment, the method comprises the step of adding a fluid in the upper portion of the volume, particularly in the lower top section.

In an embodiment, the fluid, particularly the medium is added in the upper portion of the volume after the generation of a matrix in the extended lower portion of the volume. The matrix in the extended lower portion of the volume can be overlaid with the fluid. In particular, the generated matrix can be overlaid with medium.

Further, by placing the tip of the pipette on the further circumferential step medium can be removed from the upper portion of the volume.

By placing the tip of the pipette on the further circumferential step is it prevented that the matrix generated in the extended lower portion of the volume is disturbed by means of the pipette.

In an embodiment, the medium in the upper portion of the volume is replaced by fresh culture medium at least once or several times.

A device according to the invention can be used in versatile applications. The device can be used for the easy, secure and cost-efficient generation of a microtissue and can further be used for a subsequent analysis of the microtissue. The device can be used in high-throughput approaches. Using the device and/or the method according to the invention a biological process such as cell migration, in particular cell invasion, can be analyzed in a large scale.

In the following, further features, advantages and embodiments of the present invention are explained with reference to the Figures, wherein

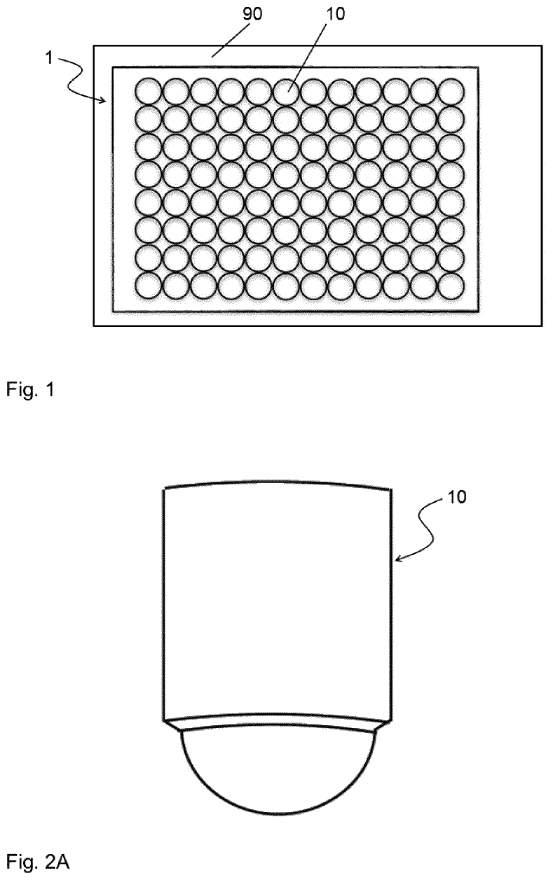

FIG. 1 shows a schematic top view of an embodiment of a device comprising 96 wells,

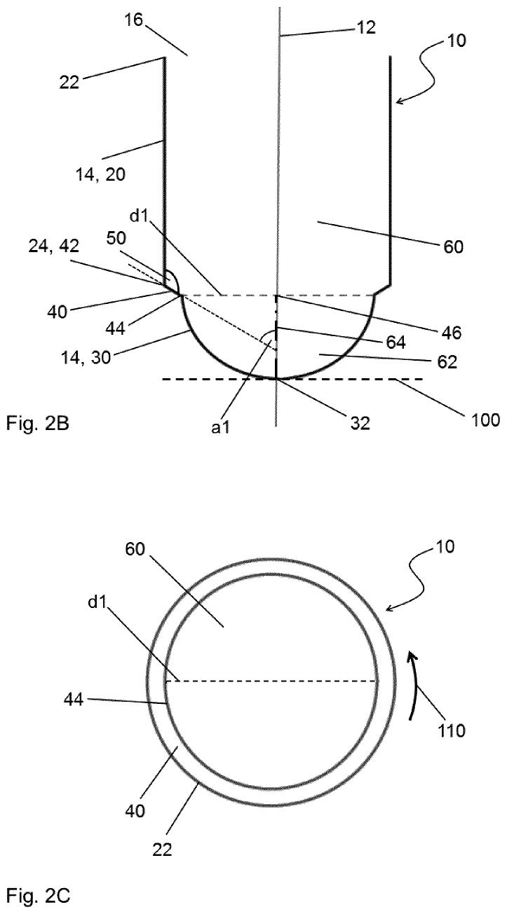

FIG. 2 shows a schematic side view (A), a schematic cross section view (B) and a schematic top view (C) of a well comprising a curved bottom section,

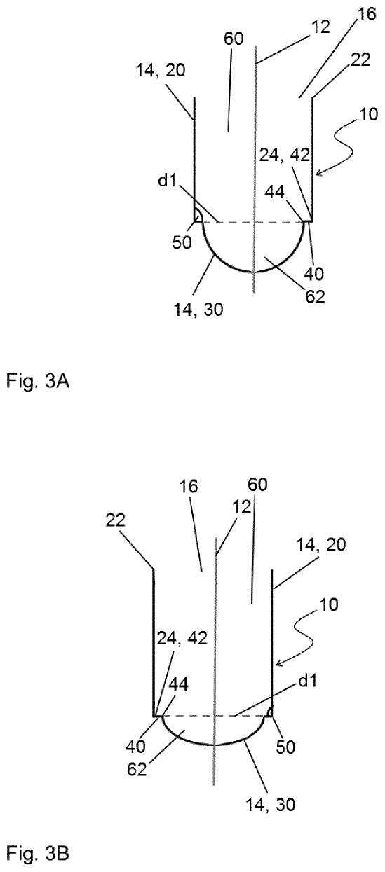

FIG. 3 shows schematic cross section views of four wells comprising a curved bottom sections (A-D),

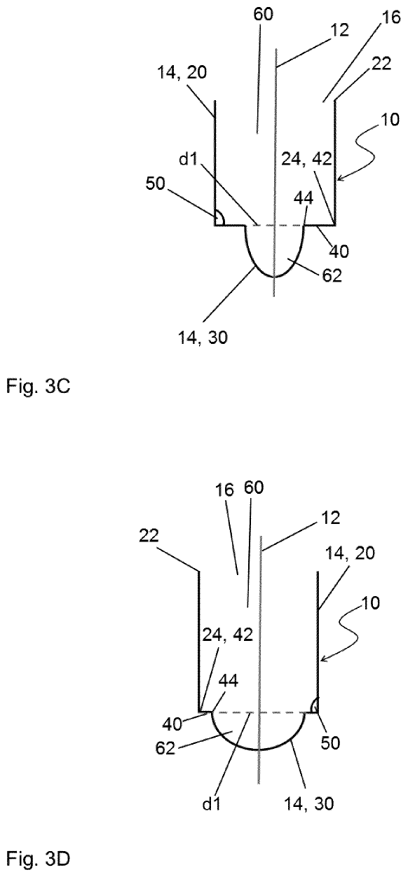

FIG. 4 shows schematic perspective views (A, C), schematic cross section views (B, D) and a schematic top view (E) of a well comprising a flat bottom,

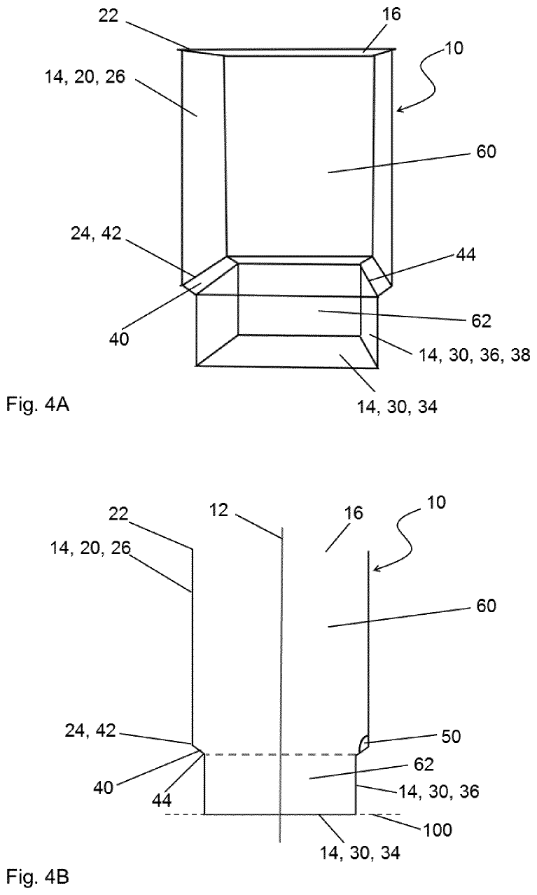

FIG. 5 shows a schematic side view of a well wherein the circumferential step has a conic shape and a tip of a pipette is placed into the well (A, B),

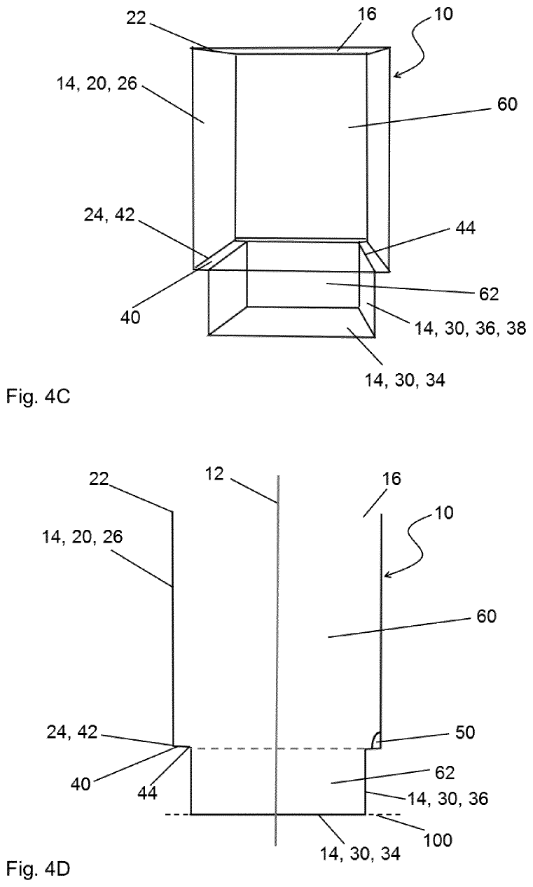

FIG. 6 shows a schematic side view of a well wherein the circumferential step has the shape of a circular ring and a tip of a pipette is placed into the well,

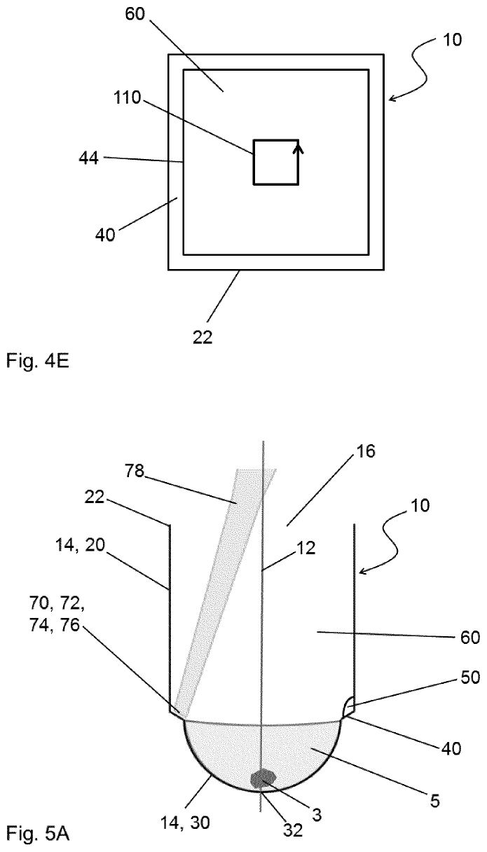

FIG. 7 shows a schematic cross section view of a well comprising a curved bottom section,

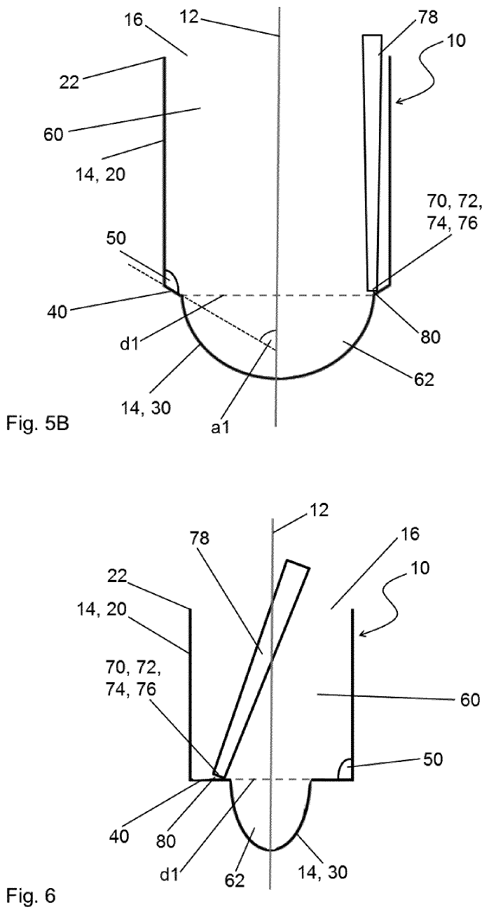

FIG. 8 shows a schematic perspective view of a well comprising a curved bottom section,

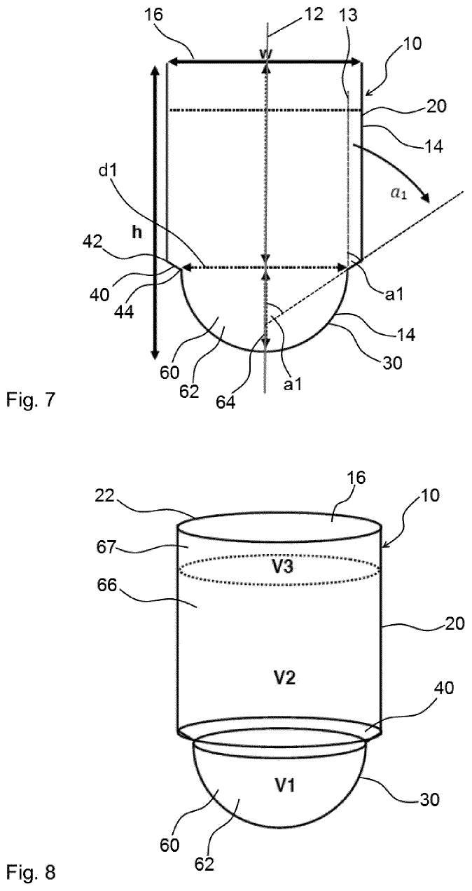

FIG. 9 shows a schematic cross section view of a well comprising a curved bottom section, a circumferential step and a further circumferential step,

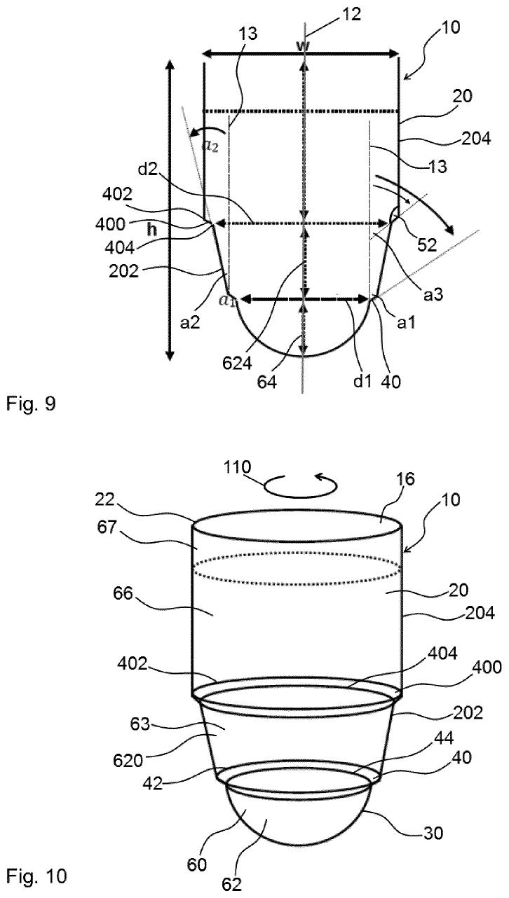

FIG. 10 shows a schematic perspective view of a well comprising a curved bottom section, a circumferential step and a further circumferential step, and

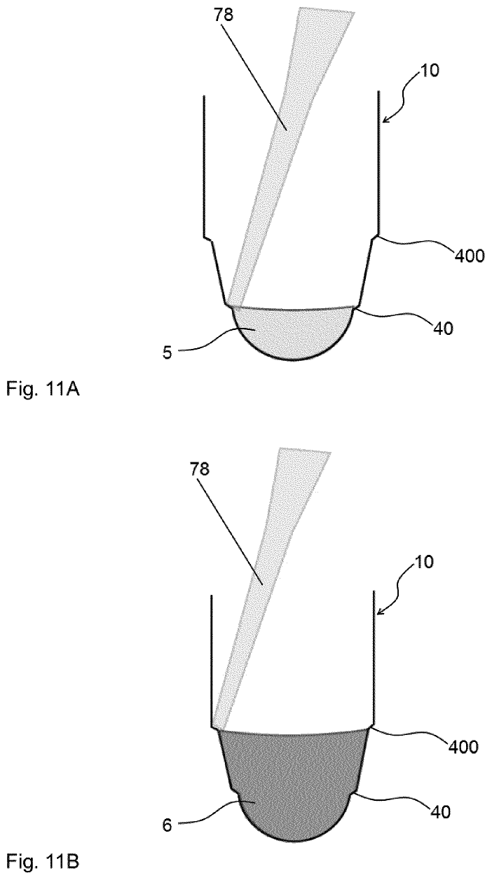

FIG. 11 shows a schematic cross section view of a well a circumferential step and a further circumferential step, wherein a tip of a pipette is put on the circumferential step (A) or the further circumferential step (B).

FIG. 1 shows a top view of a device 1 that comprises a plurality of wells 10, here as an example 96 wells 10. The device 1 can have a rectangular ground area. The wells 10 can be arranged in an array comprising e.g. eight rows wherein each row comprises e.g. 12 wells 10. Each individual well 10 can have a circular cross-sectional area. All wells 10 can be configured identically.

The device 1 can be placed on the stage of a microscope 90, in particular on the stage of a fluorescence microscope.

A well 10 can comprise a bottom section 30 that comprises a concave curvature, hereafter also referred to as curved bottom section 30. Exemplary embodiments of a well 10 comprising a curved bottom section 30 are shown in FIGS. 2, 3, 5, 6, 7, 8, 9, 10 and 11.

In an alternative embodiment, a well 10 can comprise a bottom section 30 that comprises a flat bottom 34; hereafter also referred to as flat bottom section 30 (shown in FIG. 4).

As can be seen in FIGS. 2 to 11, the well 10 can extend along a central axis 12. The well can extend along a longitudinal axis 13. The longitudinal axis 13 can be the central axis 12. The well 10 can be rotationally symmetrical with respect to the central axis 12 (FIGS. 2, 3, 5-11).

The well 10 can comprise an inner surface 14 that faces a volume 60 for receiving a biological sample 3. The inner surface 14 comprises a top section 20, a bottom section 30 and a circumferential step 40 that connects the top section 20 and the bottom section 30. The circumferential step 40 can be a rim, particularly a circumferential rim.

The top section 20 can extend parallel or coaxially to the central axis 12. In an embodiment, the top section 20 can have the shape of an open cylinder. In an embodiment, the top section 20 can have the shape of an open circular right cylinder (FIGS. 2, 3, 5, 6, 7, 8). In another embodiment, the top section 20 can comprise four rectangular sides 26 wherein each two adjacent rectangular sides 26 can be arranged perpendicular to each other (FIG. 4). All four rectangular sides 26 can be configured identically, in particular, each rectangular side 26 can have the same width and height. In a further embodiment, the top section 20 can taper towards the bottom section 30 (FIGS. 9-11).

The upper edge of the top section 22 can delimit an opening of the well 16. The opening of the well 16 can have a circular shape (FIGS. 2, 3, 5-11). Alternatively, the opening of the well 16 can have a rectangular shape, in particular a squared shape (FIG. 4). The volume 60 can be accessed via the opening of the well 16. The lower edge of the top section 24 can border the circumferential step 40. The lower edge of the top section 24 can abut the circumferential step 40.

The circumferential step 40 can extend along the entire circumferential direction 110 and can form a circumferential surface (FIGS. 2C, 4E, 8, 10). Further, the circumferential step 40 can comprise an outer edge 42 and an inner edge 44. The outer edge 42 can be adjacent to the lower edge of the top section 24. The lower edge of the top section 24 can abut the outer edge 42 of the circumferential step 40.

The inner edge 44 of the circumferential step 40 borders the bottom section 30.

The inner edge 44 can have the shape of a circle comprising a center of that circle 46 (FIGS. 2, 3, 5-11). The well 10 can be configured such that the center of the circle determined by the inner edge 46 is located on the central axis 12. The inner edge 44 can have a diameter d1 (FIGS. 2, 3, 5, 6, 7, 9).

In an embodiment the circle described by the inner edge 44 of the circumferential step can have the diameter d1 in a range from 3.5 mm to 6.5 mm, in particular from 5 mm to 6 mm. In an embodiment, the diameter d1 of the circle described by the inner edge 44 of the circumferential step can be 5.94 mm.

In another embodiment, the inner edge 44 can have the shape of a square (FIG. 4). The square described by the inner edge 44 of the circumferential step can comprise a diagonal in a range from 4 mm to 10 mm, in particular from 4.24 mm to 8.5 mm.

The center of the square determined by the inner edge 46 can be located on the central axis 12.

The top section 20 and the circumferential step 40 can enclose a first angle 50. In an embodiment, the first angle 50 can be an obtuse angle (FIGS. 2B, 4A, 4B, 5A, 5B). In particular, the first angle 50 can be in a range between 115° and 125°.

In an embodiment, the circumferential step 40 encloses an acute angle a1 with the longitudinal axis 13 (see e.g. FIGS. 2B, 5B, 7). The circumferential step 40 can enclose the acute angle a1 with the central axis 12 (see e.g. FIGS. 2B, 5B, 7).

The circumferential step 40 can taper in a direction from the outer edge 42 to the inner edge 44 (FIGS. 2, 4A, 4B, 5A, 5B). The diameter d1 related to the inner edge 44 of the circumferential step 40 can be smaller than a diameter related to the outer edge 42 of the circumferential step 40. In an embodiment, the circumferential step 40 comprises a conical shape (FIGS. 2B, 5A, 5B, 8, 10). In another embodiment, the circumferential step 40 can have the shape of a frustum, in particular of a square frustum (FIGS. 4A, 4B).

In an embodiment, the first angle 50 can be an angle of 90° (FIGS. 3A-D; 4C, 4D, 6). This means that the top section 20 and the circumferential step 40 are arranged perpendicular to each other.

The inner edge 44 of the circumferential step borders the bottom section 30. The inner edge 44 marks a predefined lower portion of the volume 62 (indicated by a dashed grey line in FIGS. 2 to 6). Further, the lower portion of the volume 62 is confined by the bottom section 30.

The lower portion of the volume 62 can be between 10 μl and 120 μl, in particular between 15 μl and 50 μl, in particular 30 μl.

According to an embodiment, the lower portion of the volume 62 is at least 10% of the volume 60. According to an embodiment, the lower portion of the volume 62 is 20% of the volume 60.

The volume 60 can comprise the lower portion of the volume 62 and an upper portion of the volume 66. In an embodiment, the volume 60 consist of the lower portion of the volume 62 and the upper portion of the volume 66.

In an embodiment, the upper portion of the volume 66 comprises a topmost portion 67 of the volume 60.

In an embodiment, the bottom section 30 is a curved bottom section 30 (FIGS. 2, 3, 5-11). The curved bottom section 30 can comprise a lowest point 32. The lowest point 32 is characterized in that the distance from the lowest point 32 to the opening of the well 16 parallel to the central axis 12 is greater than the distance from any other point of the bottom section 30 to the opening 16 parallel to the central axis 12. In an embodiment, the lowest point 32 is located on the central axis 12.

The lower portion of the volume 62 can be characterized by a height of the lower portion 64 (FIG. 7, 9; also indicated by the dot-and-dashed line in FIG. 2B) wherein the height of the lower portion 64 is the distance from the lowest point 32 to the center of the circle determined by the inner edge 46. The height of the lower portion 64 can be in the range from 1 mm to 4 mm, in particular from 2 mm to 3 mm. In an embodiment, the height of the lower portion 64 can be 2.97 mm.

The bottom section 30 can comprise a concave curvature. In particular the bottom section 30 can be curved concavely. This means that the distance between the bottom section 30 and a virtual first plane 100 that comprises the lowest point 32 and that extends perpendicular to the central axis 12, increases with increasing distance from the lowest point 32.

In an embodiment, the bottom section 30 can be hemispherical (FIG. 3A). In another embodiment, the bottom section 30 can have the shape of a segment of a spheroid (FIG. 3B, C). In an embodiment, the bottom section 30 can comprise the shape of a segment of an oblate, i.e. flattened, semi-spheroid (FIG. 3B). In an alternative embodiment, the bottom section 30 can comprise the shape of a segment of a prolate, i.e. elongated, semi-spheroid (FIG. 3C). In another embodiment the bottom section 30 can have the shape of a spherical segment (FIG. 2, FIG. 3D).

In an alternative embodiment, the bottom section 30 can be a flat bottom section 30 (FIG. 4). A flat bottom section 30 can comprise a flat bottom 34 that extends in a plane, in particular in the first plane 100. In addition, the flat bottom section 30 can comprise a circumferential lateral portion 36 wherein the lateral portion 36 can comprise four rectangular sides 38. Each two adjacent rectangular sides 38 of the bottom section 30 can be arranged perpendicular to each other. The lateral portion 36 can be connected to the bottom 34. Further, the lateral portion 36 can connect the bottom 34 and the circumferential step 40. In an embodiment, the lateral portion 36 can extend perpendicular to the bottom 34.

A height of the lower portion of a well comprising a flat bottom section 30 can be in the range from 1 mm to 6 mm.

A top view of a well 10 (FIG. 2C) illustrates that a well 10 can have a circular cross section. In an embodiment, the diameter of the circle determined by the top section 20, in particular by the upper edge of the top section 22, is greater than the diameter d1 of the circle determined by the inner edge 44 of the circumferential step 40. The diameter of the circle that is determined by the upper edge of the top section 22 can be in the range from 6 mm to 7.5 mm, in particular between 6.5 mm and 7 mm. In an embodiment, the diameter of the circle that is determined by the upper edge of the top section 22 can be 6.94 mm.

In another embodiment, the well 10 can have a squared cross section (FIG. 4E). The diagonal of the square determined by the upper edge 22 can be in the range from 7 to 10 mm.

FIG. 5 and FIG. 6 as well as FIG. 11 show cross section views of a well 10 wherein a tip of a pipette 70 is placed into the well 10. In FIG. 5A and FIG. 5B, a well 10 comprising an obtuse first angle 50 is shown. In FIG. 6, a well 10 comprising a right first angle 50 is presented. The bottom section 30 can be a curved bottom section 30 that comprises the shape of a segment of a sphere (FIG. 5A and FIG. 5B) or a spheroid (FIG. 6).

A biological sample 3 can be placed into the volume 60 (FIG. 5A). In particular, the biological sample 3 can be located in the lower portion of the volume 62, in particular in the lower portion of the volume 62 close to the lowest point 32.

The lowest point 32 is located on the central axis 12. Hence, the biological sample 3 is located centrally. If the biological sample 3 is located centrally, it is visually easy to access. This is an advantage for a further analysis of the biological sample 3, in particular for the inspection of the biological sample by means of a microscope.

Further, the predefined lower portion of the volume 62 can be filled with a solution, e.g. a culture medium 5 (FIG. 5A). The circumferential step 40 and the lower portion 62 of the volume 60 can be configured for growing spheroids and medium exchange.

The circumferential step 40 can act as a stop for the tip of the pipette 70. This includes that the circumferential step 40 can control a depth the pipette 78, in particular the tip of the pipette 70, can be inserted into the respective well 10. The tip of the pipette 70 comprises a face side 76 that can comprise an edge of the pipette 74 that delimits an opening of the pipette 72. The tip of the pipette 70 can be placed in the well 10 such that the face side 76, in particular the edge of the pipette 74, can touch the circumferential step 40.

The opening of the pipette 72 and the circumferential step 40 can enclose a second angle 80. This means that the plane in that the face side 76 extends and the circumferential step 40 can enclose a second angle 80. The tip of the pipette 70 can be arranged such that the edge of the pipette 74 can be in contact with the circumferential step 40 and such that the second angle 80 is an acute angle (FIG. 5B, FIG. 6). In particular, the second angle 80 can be in the range from 3° to 60°, in particular from 5° to 45°.

In FIG. 5B the first angle 50 is an obtuse angle and the tip of the pipette 70 is arranged such in the well 10 that the opening of the pipette 72 extends perpendicular to the central axis 12 such that the second angle 80 is an acute angle.

In FIG. 6 the first angle 50 is a right angle and the tip of the pipette 70 is arranged such in the well 10 that the face side 76 extends with in inclination with respect to the central axis 12. This means that the tip of the pipette 70 is arranged such that the opening of the pipette 72 does not extend perpendicular to the central axis 12. The second angle 80 is an acute angle.

In FIG. 5A, the first angle 50 is an obtuse angle and the tip of the pipette 70 is arranged such in the well 10 that the face side 76 extends with in inclination with respect to the central axis 12. In particular the tip of the pipette 70 is arranged such in the well 10 that the face side 76 extends parallel to the circumferential step 40.

In FIG. 9 a cross section view of a well 10 is illustrated that comprises a circumferential step 40 and a further circumferential step 400. FIG. 10 shows a perspective view of a well 10 comprising the circumferential step 40 and the further circumferential step 400.

The circumferential step 40 connects the top section 20 and the bottom section 30. The top section 20 can comprise a lower top section 202 and an upper top section 204. The upper top section 204 can be connected with the lower top section 202 via the further circumferential step 400. The lower top section 202 can connect the circumferential step 40 and the circumferential step 400.

In an embodiment, the upper top section 204 has a cylindrical shape (FIGS. 9, 10). Along the central axis 12, the upper top section 204 can extend parallel to the central axis 12.

In an alternative embodiment, the upper top section has a conical shape, wherein the upper top section tapers towards the further circumferential step.

According to an embodiment, the lower top section 202 can have a conical shape. The lower top section 202 can taper in direction from the further circumferential step 400 towards the circumferential step 40. The lower top section 202 can enclose a tapering angle a2 with the central axis 12. The lower top section 202 can enclose a tapering angle a2 with the longitudinal axis 13. The tapering angle a2 can be an acute angle.

In the illustrated embodiment, the tapering angle a2 is smaller than the acute angle a1. The tapering angle a2 can be smaller than the further acute angle a3. In an embodiment, the tapering angle a2 is smaller than the acute angle a1 and the further acute angle a3.

In an embodiment, in direction towards the bottom section 30, the lower top section 202 declines steeper than the circumferential step 40. In direction towards the bottom section 30, the lower top section 202 can decline steeper than the further circumferential step 400.

In an alternative embodiment, the lower top section has a cylindrical shape.

The further circumferential step 400 can enclose a further acute angle a3 with the longitudinal axis 13 and/or with the central axis 12. In an embodiment according to the invention, the acute angle a1 and the further acute angle a3 are equal. The circumferential step 40 and the further circumferential step 400 can run in parallel.

In an embodiment, the further circumferential step 400 extends along the entire circumferential direction 110 and can form a further circumferential surface (FIG. 10).

The further circumferential step 400 can comprise a further outer edge 402 and a further inner edge 404. The further outer edge 402 can be adjacent to a lower edge of the upper top section 204.

The further inner edge 404 can have a circular shape with a further diameter d2. The diameter d2 can be greater than the diameter d1 (determined by the inner edge 44 of the circumferential step 40).

The upper top section 204 and the further circumferential step 400 can enclose a second angle 52. In an embodiment, the second angle 52 is an obtuse angle (FIG. 9).

In an embodiment, the further circumferential step 400 encloses an acute angle a3 with the longitudinal axis 13 (FIG. 9). The further circumferential step 400 can enclose the acute angle a3 with the central axis 12 (FIG. 9).

The further circumferential step 400 can taper in a direction from the further outer edge 402 to the further inner edge 404 (FIGS. 9, 10). The diameter d2 related to the further inner edge 404 of the further circumferential step 400 can be smaller than a diameter related to the further outer edge 402 of the further circumferential step 400. In an embodiment, the further circumferential step 400 comprises a conical shape (FIG. 10). With respect to the upper top section 204, the further circumferential step 400 can be directed inwards, i.e. towards the central axis 12. The upper portion of the volume 66 can comprise the topmost portion 67 of the volume 60.

The further inner edge 404 can mark a predefined extended lower portion 63 of the volume 60.

The predefined extended lower portion 63 of the volume 60 can comprise the lower portion 62 of the volume 60 and a further portion 620 of the volume 60. In particular, the predefined extended lower portion of the volume 63 can consist of the lower portion 62 of the volume 60 and the further portion 620 of the volume 60 (FIG. 10).

Further, the predefined extended lower portion 63 of the volume 60 can be confined by the bottom section 30 and the lower top section 202.

According to an embodiment, the predefined extended lower portion 63 of the volume 60 is at least 5% of the volume 60. According to an embodiment, the predefined extended lower portion 63 of the volume 60 is 10% of the volume 60.

The volume 60 can comprise the predefined extended lower portion 63 and an upper portion of the volume 66. In an embodiment, the volume 60 consist of the extended lower portion 63 of the volume 60 and the upper portion of the volume 66.

Along the central axis 12, the circumferential step 40 and the further circumferential step 400 can be arranged distant to each other. Along the central axis 12, a distance 624 can be between the circumferential step 40 and the further circumferential step 400. The further portion 620 of the volume 60 can be confined by the distance 624. The further portion 620 of the volume 60 can be confined by the tapering angle a2. The further portion 620 of the volume 60 can be confined by the diameter d1. The further portion 620 of the volume 60 can be confined by the diameter d2.

The predefined extended lower portion 63 of the volume 60 determined by the lower portion 62 of the volume 60, the distance 624, the tapering angle a2, the diameter d1 and or the diameter d2.

In an embodiment, the further circumferential step 400 is arranged in a precise chosen distance 624 from the circumferential step 40. The dimensions of the well can define different volumes in the respective well 10 separated by the steps 40, 400. In particular, the lower portion 62, the extended lower portion 63, the further portion 620 and the upper portion 66 can be defined. The ratios between the lower portion 62, the extended lower portion 63, the further portion 620 and the upper portion 66 of the volume 60 can be predefined. The circumferential step 40 and the further circumferential step 400 can be configured and arranged such that the predefined ration between the volumes, in particular the lower portion 62, the extended lower portion 63, the further portion 620 and the upper portion 66 of the volume 60 are met. This can advantageously provide mixing predefined volume of solution with a particular concentration with a further predefined volume of polymer at a predefined further concentration. After polymerization, solidified matrix can be overlaid with medium. The further circumferential step 400 can facilitate the medium exchange.

The predefined lower portion of the volume 62 can be filled with a solution, e.g. a culture medium 5 (FIG. 11A). The circumferential step 40 can act as a stop for the tip of the pipette 70.

The extended lower portion 63 of the volume 60 can be filled with a solution, e.g. a culture medium 5 (FIG. 11B). The further circumferential step 400 can act as a stop for the tip of the pipette 70.

According to an embodiment, the further circumferential step 400 can be arranged and configured for medium exchange of matrix-embedded cultures. The further circumferential step 400 can be configured to prevent the sucking-up of matrix during medium exchange.

The well 10 can be configured such that a fluid 5 can be removed from the volume 60 such that the fluid 5 remains in the predefined lower portion 62 of the volume 60 exclusively (see e.g. FIG. 11A). In particular, the tip 70 of the pipette 78 can be positioned on the circumferential step 40 and fluid 5 can be removed. The fluid 5 will remain in the predefined lower portion 62 of the volume 60.

The well 10 can be configured such that the fluid remains in the extended lower portion 63 of the volume 60 exclusively. In other words this means that the well can be configured such that fluid can be removed from the upper portion 66 of the volume 60. In particular, the tip 70 of the pipette 78 can be positioned on the further circumferential step 400 and fluid can be removed. The fluid will remain in the extended lower portion 63 of the volume 60.

The well 10 can be configured such that a polymerised matrix 6 remains in the extended lower portion 63 of the volume 60 exclusively.

In the following a further aspect of the present invention as well as embodiments thereof are stated as items, wherein the reference numerals in parentheses relate to the Figures. These items may also be formulated as claims of the present invention.

Item 1: A method for propagating biological samples (3) using a device (1) according to one of the preceding claims, comprising the steps of: providing a suspension of cells (3) in a culture medium (5) in a plurality of wells (10) of the device (1); incubating the cells (3); placing a tip of a pipette (70) on the respective circumferential step (40); and removing culture medium (5) by means of the pipette (78) such that culture medium (5) and cells (3) remain in a lower portion of the volume (62) of the respective well (10).

Item 2: The method according to item 1, wherein a portion of the culture medium (5) is replaced by fresh culture medium (5) at least once or several times.

Item 3: The method according to item 1 or 2, wherein the method comprises the step of: adding a compound to the culture medium in the respective well (10) such that the culture medium (5) polymerizes.

Item 4: The method according to one of the items 1 to 3, wherein the method further comprises the step of: adding a solution to the culture medium in a plurality of wells (10) wherein the solution is a control medium (5), or wherein the solution comprises one of: a stimulating compound for stimulating the biological sample (3), an inhibiting compound for inhibiting the biological sample (3), a combination of a stimulating and an inhibiting compound.

Item 5: The method according to one of the items 1 to 4, wherein the method further comprises the steps of: adding a fluorescent dye to the culture medium in a plurality of wells (10); putting the device (1) on the stage of a microscope (90), in particular a fluorescence microscope; and acquiring images of cells (3) in the respective well (10) by means of the microscope.

Item 6: The method according to one of the items 1 to 5, wherein the tip of the pipette (70) comprises an opening (72), wherein the tip of the pipette (70) is placed such that the opening of the pipette (72) and the circumferential step (40) enclose a second angle (80), wherein the second angle (80) is in the range from 0° to 75°, in particular in the range from 5° to 45°.

### LIST OF REFERENCE NUMERALS

1 device
3 biological sample or cells
5 medium
6 matrix
10 well
12 central axis
13 longitudinal axis
14 inner surface
16 opening of the well
20 top section
22 upper edge of the top section
24 lower edge of the top section
26 rectangular side of the top section
30 bottom section
32 lowest point
34 flat bottom
36 lateral portion
38 rectangular side of the bottom section
40 circumferential step
42 outer edge
44 inner edge
46 center of the circle determined by the inner edge
50 first angle
52 second angle
60 volume
62 lower portion of the volume
63 extended lower portion of the volume
64 height of the lower portion
66 upper portion of the volume
67 topmost portion of the volume
70 tip of a pipette
72 opening of a pipette
74 edge of a pipette
76 face side of a pipette
78 pipette
80 second angle
90 stage of a microscope
100 first plane
110 circumferential direction
202 lower top section
204 upper top section
400 further circumferential step
402 further outer edge
404 further inner edge
620 further portion of the volume
624 distance

1. A device (1) for receiving a biological sample (3) comprising:
a plurality of wells (10),
wherein each well (10) comprises an inner surface (14) facing a volume (60) for receiving a biological sample (3),
wherein the inner surface (14) comprises a top section (20) and a bottom section (30),
characterized in that
the top section (20) and the bottom section (30) are connected via a circumferential step (40) forming a stop for a tip of a pipette (70).

2. The device (1) according to claim 1, characterized in that the circumferential step (40) comprises an inner edge (44) marking a predefined lower portion of the volume (62).

3. The device (1) according to claim 1, characterized in that the bottom section (30) comprises a concave curvature.

4. The device (1) according to claim 1, characterized in that the bottom section (30) comprises a spherical curvature.

5. The device (1) according to claim 1, characterized in that the bottom section (30) comprises a flat bottom (34).

6. The device (1) according to claim 5, characterized in that the bottom section (30) comprises a lateral portion (36) connected to the bottom (34).

7. The device (1) according to claim 6, characterized in that the lateral portion (36) is cylindrical.

8. The device (1) according to claim 6, characterized in that the lateral portion (36) comprises four rectangular sides (38).

9. The device (1) according to claim 1, characterized in that a first angle (50) enclosed by the top section (20) and the circumferential step (40) is in the range from 90° to 135°, in particular from 100° to 130°, in particular from 115° to 125°, wherein particularly the first angle (50) is 120°.

10. The device (1) according to claim 1, characterized in that each well (10) extends along a longitudinal axis (13), wherein the circumferential step (40) is configured such that the circumferential step (40) encloses an acute angle (a1) with the longitudinal axis (13).

11. The device (1) according to claim 10, characterized in that the acute angle (a1) lies in the range from 5° to 85°, in particular in the range from 15° to 75°, in particular in the range from 30° to 60°.

12. The device (1) according to claim 1, characterized in that each well (10) extends along a central axis (12) and is rotationally symmetrical with respect to the central axis (12).

13. The device (1) according to claim 1, characterized in that each well (10) comprises or is formed out of a transparent material.

14. The device (1) according to claim 1, characterized in that the device (1) is configured to be arranged on a stage of a microscope (90).

15. The device (1) according to claim 1, characterized in that the bottom section (30) comprises a coating for reducing adhesion of cells.

16. The device (1) according to claim 1, characterized in that the device (1) comprises at least 6, particularly at least 10, particularly at least 20, particularly at least 30, particularly at least 40, particularly at least 50, particularly at least 60, particularly at least 70, particularly at least 80, particularly at least 90, particularly at least 96 wells (10), wherein particularly the plurality of wells (10) forms an array of wells.

17. The device (1) according to claim 1, characterized in that the top section (20) comprises a lower top section (202) and an upper top section (204), wherein the lower top section (202) and the upper top section (204) are connected via a further circumferential step (400).

18. The device (1) according to claim 17, characterized in that the further circumferential step (400) encloses a further acute angle (a3) with the longitudinal axis (13), in particular wherein the further acute angle (a3) is between 5° and 85°, in particular between 15° and 75°, in particular between 30° and 60°.

19. The device (1) according to claim 17, characterized in that the further circumferential step (400) and the circumferential step (40) run in parallel to each other.

20. The device (1) according to claim 17, characterized in that the further circumferential step (400) comprises a further inner edge (404) marking a predefined extended lower portion (63) of the volume (60).

21. The device (1) according to claim 2, characterized in that the predefined lower portion of the volume (62) is at least 10% of the volume (60), in particular at least 15% of the volume (60), in particular at least 20% of the volume (60).

22. The device (1) according to claim 17, characterized in that the predefined lower portion of the volume (62) is at least 5% of the volume (60), in particular at least 7.5% of the volume (60), in particular at least 10% of the volume (60).

23. The device (1) according to claim 20, characterized in that the predefined extended lower portion (63) of the volume (60) is at least 20% of the volume (60), in particular at least 30% of the volume (60), in particular at least 40% of the volume (60).

24. The device (1) according to claim 1, characterized in that the top section (20) tapers towards the circumferential step (40).

25. A method for propagating biological samples (3) using a device (1) according to claim 1, comprising the steps of:
providing a suspension of cells (3) in a culture medium (5) in a plurality of wells (10) of the device (1),
incubating the cells (3),
placing a tip of a pipette (70) on the respective circumferential step (40), and
removing culture medium (5) by means of the pipette (78) such that culture medium (5) and cells (3) remain in a lower portion of the volume (62) of the respective well (10).
